(19) United States
(12) Patent Application Publication
(10) Pub. No.: US 2020/0359903 A1
(43) Pub. Date: Nov. 19, 2020

(54) COHERENCE GATED PHOTOACOUSTIC REMOTE SENSING (CG-PARS)

(71) Applicant: ILLUMISONICS INC., Edmonton (CA)

(72) Inventors: Parsin HAJI REZA, Edmonton (CA); Roger ZEMP, Edmonton (CA); Kevan BELL, Edmonton (CA)

(73) Assignee: ILLUMISONICS INC., Edmonton, AB (CA)

(21) Appl. No.: 16/753,887

(22) PCT Filed: Sep. 28, 2018
(86) PCT No.: PCT/IB2018/057585

§ 371 Date: Apr. 6, 2020

Related U.S. Application Data
(60) Provisional application No. 62/622,816, filed on Jan. 26, 2018.

(51) Int. Cl. A61B 5/00; G01N 21/17; A61B 5/145; A61B 5/0205; A61B 5/026; A61B 5/0275

(52) U.S. Cl. CPC A61B 5/0095; G01N 21/1702; A61B 5/14542; A61B 5/0205; A61B 5/0261; A61B 5/0275; A61B 5/0833; A61B 5/444; A61B 5/445; A61B 5/418; A61B 5/4255; A61B 5/413; G01N 2021/1706; A61B 5/443

(57) ABSTRACT

A coherence gated photoacoustic remote sensing system for imaging a subsurface structure in a sample with optical resolution may include an excitation beam source configured to generate an excitation beam that induces ultrasonic signals in the sample at an excitation location; an interrogation team source configured to generate an interrogation team incident on the sample at an interrogation location, a portion of the interrogation beam returning from the sample that is indicative of the generated ultrasonic signals, the interrogation beam being a low-coherent beam; an optical system that focuses the excitation beam onto the sample at an excitation location, and focuses the interrogation beam onto the sample at an interrogation location, at least the interrogation location being below the surface of and within the sample; and a low coherence interferometer that isolates a returning portion of the interrogation beam that corresponds to an interrogation event of the sample.

### CROSS-REFERENCE TO RELATED APPLICATION(S)

This patent application claims benefit of priority under 35 U.S.C. § 119 to U.S. Provisional Patent Application No. 62/622.816, filed Jan. 26, 2018, the entirety of which is incorporated herein by reference.

### FIELD

This relates to the field of optical imaging and, in particular, to a laser-based method and system for non-contact imaging of biological tissue in vivo, ex vivo, or in vitro.

### BACKGROUND

The entireties of the U.S. Patents and Patent Publications set forth herein are expressly incorporated by reference.

Photoacousfic imaging is an emerging hybrid imaging technology providing optical contrast with high spatial resolution. Nanosecond or picosecond laser pulses fired into tissue launch thereto-elastic-induced acoustic waves, which are detected and reconstructed to form high-resolution images.

Photoacoustic imaging has been developed into multiple embodiments, primarily including photoacoustic tomography (PAT), photoacoustic microscopy (PAM) which is sometimes referred to as acoustic-resolution photoacoustic microscopy (AR-PAM), and optical-resolution photoacoustic microscopy (OR-PAM). In PAT, signals are collected from multiple transducer locations and reconstructed to thrill, a tomographic image in a way similar to ultrasound (US) or X-ray computed tomography (CT). One of the differences between PAT and the other two modalities is that an assumption must be made about the sample in order to facilitate reconstruction; typically, this involves assuming the acoustic propagation velocity within the sample. In PAM, typically, a single element focused high-frequency ultrasound transducer is used to collect photoacoustic signals providing acoustic focusing. This transducer, along with the excitation beam may be scanned laterally about the sample to perform volumetric imaging. Both PAT and PAM are typically implemented using an unfocused excitation beam. Both modalities provide acoustic-limited resolution and have penetration depth limited by surface optical exposure limits and acoustic attenuation. OR-PAM, typically, utilizes both optical and acoustic focusing providing further improved resolution (˜3 um) at further reduced penetration depths (˜1 mm) now limited by fundamental light transport, that is, the distance which optical focus can be reasonably maintained. In all three embodiments, the acoustic signal is typically collected through an acoustically coupled transducer or other acoustic- or acousto-optic resonator. In all cases the photoacoustic signal can be recorded for various positions to form a 2D or 3D photoacoustic image representing the optical absorption in the sample at the excitation wavelength. The amplitude of the various recorded peaks implies the local optical absorption, and the relative time delay infers the depth from the time required for acoustic propagation.

Photoacoustic microscopy has shown significant potential for imaging vascular structures from macro-vessels to micro-vessels. It has also shown great promise for functional and molecular imaging, it imaging of nanoparticle contrast agents and imaging of gene expression. Multi-wavelength photoacoustic imaging has been used for spectral unmixing such as mapping of blood oxygen saturation, by using known oxy- and deoxy-hemoglobin molar extinction spectra. Since conventional photoacoustic imaging requires acoustic coupling to the sample the technique is inappropriate for many clinical applications such as wound healing, burn diagnostics, surgery, and many endoscopic procedures. Here, physical contact, coupling, or immersion is undesirable or impractical. Some non-contact photoacoustic detection strategies have been reported.

However, until recently no technique has demonstrated practical non-contact in vivo microscopy in reflection mode with confocal resolution and optical absorption as the contrast mechanism. Most previous approaches detected surface oscillations with interferometric methods which have suffered from poor sensitivity and have been ineffective for high quality in vivo imaging. One example of a low-coherence interferometry method for sensing photoacoustic signals was proposed in (Gurton et al., US Patent Publication No. 2014/0185055) to be combined with an optical coherence tomography (OCT) system, resulting in 30 μm lateral resolution. Another system is described in (Rousseau et al., US Patent Publication No. 2012/0200845) entitled “Biological Tissue Inspection Method and System”, which describes a noncontact photoacoustic imaging system for in vivo or ex vivo, non-contact imaging of biological tissue without the need for a coupling agent. Other systems use a fiber based interferometer with optical amplification to detect photoacoustic signals and form photoacoustic images of phantoms with acoustic. (not optical) resolution. However, these systems suffer from a. poor signal-to-noise ratio. Furthermore, in vivo imaging was not demonstrated, and optical-resolution excitation was not demonstrated.

A recently reported photoacoustic technology blown as photoacoustic remote sensing (PARS) microscopy (Haji Rem et al., US Patent Publication No. 2016/0113507, and Haji Reza et al., US Patent Publication No. 2017/0215738) has been able to solve many of these sensitivity issues through its detection mechanism. PARS utilizes the elasto-optic effect in which the large photoacoustic initial pressures generate nontrivial modulations in the local refractive index of a material. By co-focusing a continuous wave interrogation beam with the excitation spot, the back-reflected time-varying intensity of the interrogation beam encodes information regarding this elasto-optic modulation, which in turn implies the magnitude of the generated photoacoustic initial pressure, which is directly related to the local optical absorption in the sample at the excitation spot. PARS has thus far demonstrated improved sensitivity and resolution characteristics over conventional contact-based OR-PAM, with lateral resolutions on-par with confocal microscopy (˜600 nm). However, in some examples, depth sensitivity can be improved. Since PARS may be solely sensitive to the large initial photoacoustic pressures near the excitation spot, time-domain information is not indicative of depth. This may require three dimensional optical scanning when recording 3D volumes. Since PARS has been implemented, in some examples, using a low-coherence superluminescent diode (SLD) as the detection source, some advantages may be gained by implementing a low-coherence interferometer.

Optical coherence tomography (OCT) provides a means of capturing depth-resolved optical scattering information from a sample. This is generally accomplished by the use of low-coherence interferometry. Two common embodiments of the technique involve a time-domain approach, known as time-domain optical coherence tomography (TD-OCT), and a frequency-domain approach, known as frequency-domain optical coherence tomography (FD-OCT) or spectral-domain optical coherence tomography (SD-OCT). TD-OCT generally is implemented with a single broadband continuous-wave interrogation source which is split into a sample- and reference-path, where the total path length of the reference-path is scanned such that low-coherence interferometry is performed at various depths along the sample-path. This modality still may necessitate a 3D voxel-based scan for capturing for volumes. SD-OCT generally is implemented with either a broadband source, or a modulated frequency source, where imaging is commonly performed with a fixed reference-path length and depth information is acquired through Fourier transform of the collected spectral data. Here, volumetric scanning only may necessitate lateral scanning as full depth-resolved information is collected with a single acquisition event. There has been a great body of work within the OCT field to provide quantitative optical absorption measurement. This is the particular interest within the ophthalmic imaging community, which requires oxygen saturation measurement about the fundus of the eye. While there have been several notable works on this topic, the current approach is still incapable of direct optical absorption measurement (unlike photoacoustic modalities). Rather, optical absorption must be inferred through the use of a visible probe source which can greatly limit the penetration depth into the sample. The resulting OCT image is fit to optical extinction curves providing optical absorption. It would be beneficial to the biomedical imaging community to offer an improved optical absorption modality.

There have been several notable attempts to provide a multi-modality implementation of non-PARS-based non-contact photoacoustics and OCT. These include but are not limited to (Wang, US Patent Publication No. 2014/0185055, Johnson et al., US Patent Publication No. 2014/0275942, and Ode, U.S. Pat. No. 9,335,253). However, all of these works do not provide the same method of operation presented here in that they simply provide separately a non-contact PAT and OCT system. The proposed approach is not to be confused with previous OCT-based photoacoustic detection methods which aimed to detect propagated acoustic waves manifesting themselves as subtle oscillations at the sample outer surface. Instead the proposed approach locally detects optical-absorption-induced initial pressures directly at their sub-surface origins. Additionally, the photoacoustic component of each is specifically analogous to a PAT system in that lateral tomographic reconstruction is required and acoustic-resolution is provided.

Given these complementary properties between PARS and OCT, there would be a clear benefit towards augmenting PARS with various coherence-gated detection mechanisms. However, for reasons which will be discussed in further sections, a great deal of technical challenges arise with these implementation which are addressed in this disclosure.

### SUMMARY

According to an aspect, there is provided a coherence-gated photoacoustic remote sensing system (CG-PARS) for imaging a subsurface structure in the sample known as coherence-enhanced photoacoustic remote sensing (CEPARS) microscopy, which provides significant axial-resolution characteristics over conventional PARS. This may be accomplished through the addition of a low-coherence interferometer between the sample-path and a newly included reference-path, wherein by virtue of low-coherence interferometry, signals which are associated with path lengths significantly longer or shorter than the reference-path length (when compared with the coherence-length of the broadband interrogation source) are rejected. This may comprise an excitation beam configured to generate ultrasonic signals in the sample-path at an excitation location; an interrogation beam incident on the sample at the excitation location, a portion of the interrogation beam returning from the sample that is indicative of the generated ultrasonic signals; a single reference-path, or multiple reference-paths which may provide various phase offsets, or an optical quadrature detector; an optical combiner to compare the back-reflected sample beam with the single reference, or multiple combiners to compare the back-reflected sample beam with the multiple reference-paths; single, or multiple detectors for collecting the combined beams; and a processing unit for interpreting collected results.

According to another aspect, there is provided an endoscopic CEPARS which may provide significant axial-resolution characteristics over conventional endoscopic PARS. This may comprise of a fiber optic cable having an input end and a detection end; an excitation beam coupled to the input into the input end of the optical fiber configured to generate ultrasonic signals in the sample-path at an excitation location; an interrogation beam coupled into the input end of the optical fiber incident on the sample at the excitation location, a portion of the interrogation bean returning from the sample back along the optical fiber that is indicative of the generated ultrasonic signals; a single reference-path, or multiple reference-paths, which may provide various phase offsets; an optical combiner to compare the back-reflected sample beam with the single reference, or multiple combiners to compare the back-reflected sample beam with the multiple reference-paths; single, or multiple detectors for collecting the combined beams; and a processing unit for interpreting collected results.

According to another aspect, there is provided a CG-PARS system for imaging a subsurface structure in the sample blown as spectral-domain coherence-gate photoacoustic remote sensing (SDCG-PARS) microscopy which provides the ability to image full depth-resolved optical-absorption within a sample within a single rapid pulse-train drastically improving imaging speeds over conventional PARS, and aforementioned CEPARS. This is accomplished through the addition of a low-coherence interferometer between the sample-path and a reference-path, a detector capable of detecting the spectral content of the combined reference- and sample-paths, and the addition of a rapid (<100 ns) interrogation mechanism such as a pulsed interrogation source, a rapidly modulated continuous-wave (CW) source, photodiode array, rapid shudder, etc. This allows for acquisition of the depth-resolved scattering profile both before, and directly after the sample has undergone photoacoustic excitation. The difference between these two scattering profiles being indicative of the optical absorption. This may comprise an excitation beam configured to generate ultrasonic signals in the sample-path at an excitation location; an interrogation beam incident on the sample at the excitation location, a portion of the interrogation beam returning from the sample, where the spectrum is indicative of the generated ultrasonic signals; a reference-path which may provide various phase offsets; and optical combiner to compare the back-reflected sample beam with the reference beam; a spectrum detector, which by its own virtue, or virtue of other components is capable of short interrogation times (<100 ns); and a processing unit for interpreting collected results.

According to another aspect, there is provided an endoscopic SDCG-PARS which provides full depth-resolved acquisitions. This comprises a fiber optic cable having an input end and a detection end; an excitation beam coupled to the input into the input end of the optical fiber configured to generate ultrasonic signals in the sample-path at an excitation location; an interrogation beam coupled into the input end of the optical fiber incident on the sample at the excitation location, a portion of the interrogation beam returning from the sample back along the optical fiber where the spectrum is indicative of the generated ultrasonic signals; a reference-path which may provide various phase offsets; and optical combiner to compare the back-reflected sample beam with the reference beam; a spectrum detector, which by its own virtue, or virtue of other components is capable of short interrogation times (<100 ns); and a processing unit for interpreting collected results.

For other embodiments of CEPARS and SDCG-PARS, the excitation source may comprise of a single or multiple sources which are pulsed, or CW and modulated. Excitation sources may be narrow-band and may cover a wide range of wavelengths or broadband individually providing wider spectra. This variety of excitation spectral content provides a means of implementing absorption-contrast spectral unmixing of the various target species in a sample. The interrogation source may likewise comprise of a single or multiple sources which are pulsed, or CW and modulated. Interrogation sources may be narrow-hand and may cover a wide range of wavelengths or broadband individually providing wider spectra. This variety of interrogation spectral content provides a means of controlling the extinction (thereby the penetration) of the interrogation beam and a means of controlling the effective coherence-length which dictates the axial-resolution of the device. The optical beam combiner may comprise of an optical coupler such as a beam-splitting cube for bulk optical implementation or a fiber coupler for fiber-based implementation, or some variety of interferometer such as a bulk- or fiber-based Michelson interferometer, common path interferometer (using specially designed interferometer objective lenses), Fizeau interferometer, Ramsey interferometer, Fabry-Perot interferometer or Mach-Zehnder interferometer. Scanning of the interrogation location may be performed through optical scanning, such a galvo-mirror, MEMS mirror, resonant scanner, polygon scanner, etc., or through mechanical scanning of either the optics or the sample using single- or multiple-axes linear, or rotational stages. Extraction of relevant signal data may be performed in a solely programmatic implementation, to a relevant circuit-based processor, or through some combination of the two.

The CEPARS may be implemented using a single reference-path where phase variation is contained within a polarization state (such as circular polarization), or may require that multiple acquisitions be performed, or may be implemented using multiple reference-paths which inherently provide phase variation through the use of different path lengths. Detection of the various combined beams may be performed by some manner of optical intensity detector such as a photodiode, balanced photodiode, avalanche photodiode etc., CCD EMCCD, iCCD, CMOS, etc., or an array of aforementioned detectors.

The SDCG-PARS interrogation may be implemented using either a pulsed source or a CW source which is modulated when using some form of sample-and-hold detector array such as a CCD, EMCCD, iCCD etc., or may be implemented using a CW source when using some form of rapid optical switching such as a shutter or optical switch, or when using a higher bandwidth detector array such as a photodiode, balanced photodiode, avalanche photodiode, etc.

The CEPARS is distinct from time-domain optical coherence tomography (TD-OCT) in that it: (1) may include the use of a pulsed excitation laser, and (2) may be sensitive to optical absorption contrast. CEDARS may necessitate the use of at least two optical beams such that one acts to excite the sample and the other acts to detect perturbations in the sample. The CEPARS system may be distinct from PARS in that it may include: (1) one or more reference paths, (2) a means of separating the in-phase (sample with no delay reference) and quadrature (sample with delayed reference) beams, and (3) a means of detecting at least two of these beams.

The SDCG-PARS may be distinct from spectral-domain optical coherence tomography (SD-OCT) and PARS in that may include: (1) the use of a pulsed excitation laser (2) the use of a pulsed interrogation laser, or a rapidly modulated continuous-wave laser, or a continuous-wave laser along with the use of a gated camera exposure to detect signals on a sufficiently short timescale such that acoustic propagation is negligible, (3) a system to subtract the depth-resolved scatterer distributions before and immediately after the excitation pulse, and that it may require (4) at least two distinct interrogation events per acquisition location such that the difference between acquisitions infers depth-resolved optical absorption distribution. SDCG-PARS may necessitate the use of at least two optical beams such that one acts to excite the sample and the other acts to detect perturbations in the sample.

Other aspects will be apparent from the description and claims below. In other aspects, the aspects described herein may be combined together in any reasonable combination as will be recognized by those skilled in the art.

A coherence gated photoacoustic remote sensing system for imaging a subsurface structure in a sample with optical resolution may include an excitation beam source configured to generate an excitation beam that induces ultrasonic signals in the sample at an excitation location; an interrogation beam source configured to generate an interrogation beam incident on the sample at an interrogation location, a portion of the interrogation beam returning from the sample that is indicative of the generated ultrasonic signals, the interrogation beam being a low-coherent beam; an optical system that focuses the excitation beam onto the sample at an excitation location, and focuses the interrogation beam onto the sample at an interrogation location, at least the interrogation location being below the surface of and within the sample; and a low coherence interferometer that isolates a returning portion of the interrogation beam that corresponds to an interrogation event of the sample.

The system may include a reference beam source configured to generate a reference beam that travels along a reference path, and wherein the low coherence interferometer isolates the returning portion using the reference beam. The reference beam source is configured to generate one or more additional reference beams that are phase shifted relative to the reference beam, and wherein the low coherence interferometer isolates the returning portion using the reference beam and the one or more additional reference beams. One or more additional reference beams are phased shifted by at least one of a different path length, one or more wave plates, and one or more circulators. The one or more additional reference beams are detected either in parallel or serially with the reference beam. The excitation beam and the interrogation beam are pulsed or intensity-modulated. The excitation location and the interrogation location are each below the surface of and within the sample. At least one of the excitation location and the interrogation location are within 1 mm of the surface of the sample. At least one of the excitation location and the interrogation location are greater than 1 μm below the surface of the sample. The excitation location and the interrogation location are focal points that are at least partially overlapping. The system includes a processor that calculates an image of the sample based on the returning portion of the interrogation beam. The interrogation beam has pulses that are sufficiently short that acoustic propagation is negligible. For each detection location, the system applies an excitation beam with more than one frequency bandwidth, phase shift, or combination thereof. The optical system interrogates each interrogation location the sample in a non-excited state and after an excitation beam excites the sample. The excitation beam source is configured to generate one or more excitation beams that excites the sample with a plurality of frequencies, a plurality of bandwidths or combinations thereof.

A method of using the system may include functional imaging during brain surgery; assessing internal bleeding and cauterization verification; imaging perfusion sufficiency of organs and organ transplants; imaging angiogenesis around islet transplants; imaging of skin-grafts, imaging of tissue scaffolds and biomaterials to evaluate vascularization and/or immune rejection; imaging to aid microsurgery; or procedures for guidance to avoid cutting critical blood vessels and nerves. A method of using the system of claim may be combined with fluorescene microscopy, two-photon and confocal fluorescence microscopy, Coherent-Anti-Raman-Stokes microscopy, Raman microscopy, or Optical coherence tomography.

The method may include performing microcirculation imaging or performing blood oxygenation parameter imaging with the system.

An endoscope may include the system.

A surgical microscope may include the system.

A method of remote sensing a sample may comprise the steps of providing a coherence gated photoacoustic remote sensing system comprising an excitation beam and an interrogation beam, the interrogation beam being a low-coherent beam; causing the excitation beam to induce ultrasonic signals in the sample at an excitation location; causing the interrogation beam to interrogate the sample at an interrogation location, wherein a portion of the interrogation beam returns from the sample that is indicative of the generated ultrasonic signals, the interrogation location being below the surface of and within the sample; using a low coherence interferometer to isolate a returning portion of the interrogation beam to achieve an interrogation event of the sample.

The method further comprises providing a reference beam that travels along a reference path, and wherein the low coherence interferometer isolates the returning portion using the reference beam. The method further comprises the step of providing one or more additional reference beams that are phase shifted relative to the reference beam, and wherein the low coherence interferometer isolates the returning portion using the reference beam and the one or more additional reference beams. The one or more additional reference beams are phased shifted by at least one of a different path length, one or more wave plates, and one or more circulators. The one or more additional reference beams are detected either in parallel or serially with the reference beam. The excitation beam and the interrogation beam are pulsed or intensity-modulated. The excitation location and the interrogation location are each below the surface of and within the sample. At least one of the excitation location and the interrogation location are within 1 mm of the surface of the sample. At least one of the excitation location and the interrogation location are greater than 1 μm below the surface of the sample. The excitation location and the interrogation location are focal points that are at least partially overlapping. The method thither comprises the step of calculating an image of the sample based on the returning portion of the interrogation beam. The interrogation beam has pulses that are sufficiently short that acoustic propagation is negligible. For each detection location, the excitation beam is operated to provide with more than one frequency, bandwidth, phase shift, or combination thereof. The method further comprises the step of interrogating each interrogation location in a non-excited state and after the excitation beam excites the sample. The excitation beam comprises one or more excitation beams that excites the sample with a plurality of frequencies, a plurality of bandwidths or combinations thereof.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

In this patent document, the word “comprising” is used in its non-limiting sense to mean that items following the word are included, but items not specifically mentioned are not excluded. A reference to an element by the indefinite article “a” does not requires that there be one and only one of the elements.

The scope of the following claims should not be limited by the preferred embodiments set forth in the examples above and in the drawings, but should be given the broadest interpretation consistent with the description as a whole.

FIG. 1 depicts a schematic overview of the excitation pathway.

FIG. 2 depicts a schematic overview of the interrogation pathway.

FIG. 3 depicts a schematic view implementation of optical sources.

FIG. 4 depicts a schematic view of yet another implementation of optical sources.

FIG. 5 depicts a schematic view of an implementation of a beam combiner.

FIG. 6 depicts a schematic view of vet another implementation of a beam combiner.

FIG. 7 is a graphical illustration of the PARS mechanism.

FIG. 8 is a graphical illustration of CEPARS signals.

FIG. 9 depicts an imaging process flow diagram for CEPARS.

FIG. 10 is a schematic view of an example system layout for a CEPARS (Parallel).

FIG. 11 is a schematic view of another example system layout for a CEPARS (Parallel).

FIG. 12 is a schematic view of yet another example system layout for a CEPARS (Serial).

FIG. 13 is a schematic view of an endoscopic example system layout for a CEPARS.

FIG. 14 is a graphical illustration of an outline of the SDCG-PARS detection mechanism, primarily highlighting the relative time with winch key process is are carried out.

FIG. 15 is a graphical illustration and enlargement of an example of a SDCG-PARS spectrum both before and after photoacoustic excitation.

FIG. 16 is an imaging process flow diagram for SDCG-PARS

FIG. 17 is a schematic view of an example system layout for a SDCG-PARS.

FIG. 18 is a schematic view of an example endoscopic system layout for a SDCG-PARS

FIG. 19 is a schematic view of a system layout for a CEPARS with a quadrature interferometer.

### DETAILED DESCRIPTION

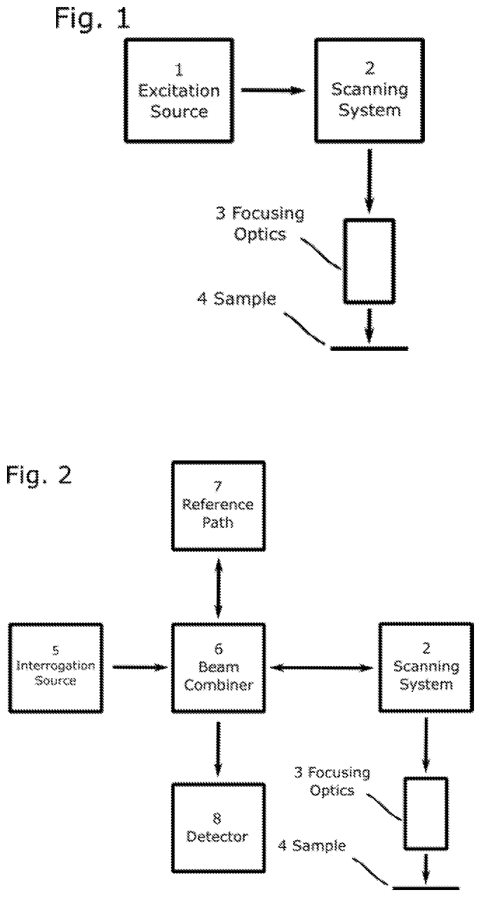

FIG. 1 shows a high-level overview of the excitation path. This primarily consists of an optical excitation source (1), an optical scanning system (2), and focusing optics (3) such as an objective lens which focuses the light onto the sample (4). The purpose of the excitation path is to direct the excitation source onto the sample to produce photoacoustic excitation within the sample.

FIG. 2 shows a high-level overview of the interrogation path. In general this consists of an optical interrogation source (5), an optical combiner (6), an optical reference path (7), an optical detector (8), and is directed onto the same optical scanning system (2), focusing optics (3), and sample (4) as in FIG. 1. The primary purpose of the interrogation path is to direct a portion of the interrogation source onto the sample, another portion being directed to the reference path to provide a desired reference path length, then to combine the beam from the sample path and the reference path as to perform low-coherence interferometry at the beam combiner. These combined optical signals are then processed appropriately at the detector to extract desired information.

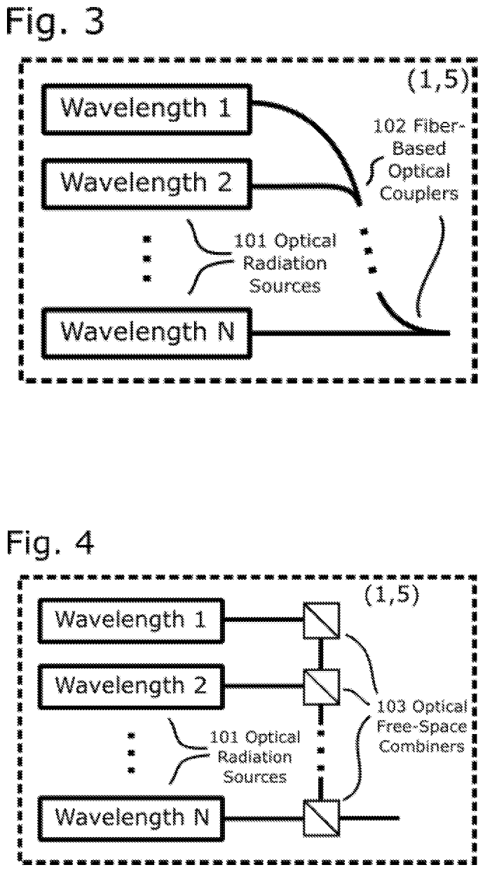

FIG. 3 shows one possible implementation of the (1) excitation source, or the (5) interrogation source which consists of one or more pulsed or modulated optical radiation sources (101) of one or more optical wavelength (1,2, . . . , N) which are fiber coupled (102) together at their respective outputs. The optical fibers may be of any type such as multimode, single mode, polarization maintaining, nonlinear, etc.

FIG. 4 shows another possible implementation of the (1) excitation source, or the (5) interrogation source which consists of one or more pulsed or modulated optical radiation sources (101) of one or more optical wavelengths (1,2, . . . , N) which are coupled together through free-space optics (103) such as beam combiners or dichroic mirrors.

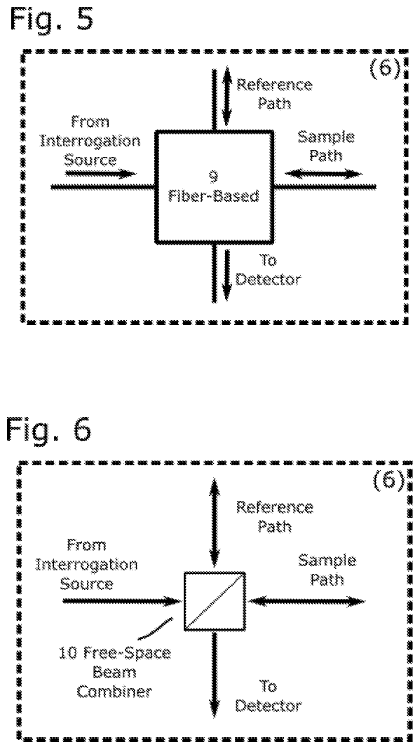

FIG. 5 shows one possible implementation of the (6) beam combiner which consists of a (11) fiber-based device such as a fiber-based interferometer or fiber-based coupler.

FIG. 6 shows another possible implementation of the (6) beam combiner which consists of a (10) free-space optical beam combiner in a Michelson interferometer layout. Note that other free-space interferometer layouts may be used such as common path interferometer (using specially designed interferometer objective lenses), Fizeau interferometer, Ramsey interferometer, Fabry-Perot interferometer and MachZehnder interferometer.

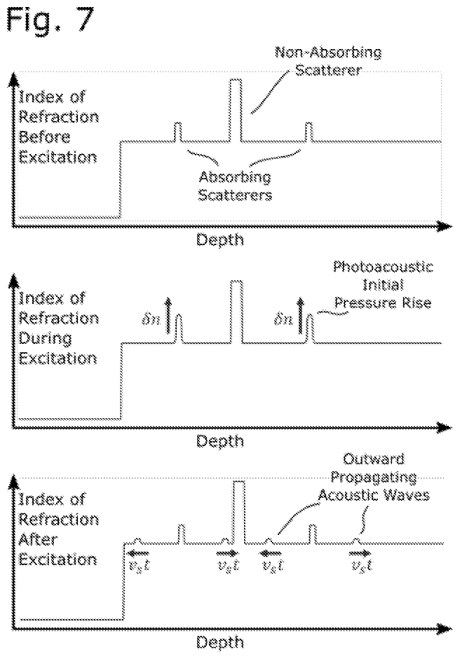

FIG. 7 highlights aspects of the PARS mechanism. Upon absorption of a sufficiently short excitation pulse (such that thermal and stress confinement conditions are met, typically shorter than 100 ns) rapid heating will occur proportional to the local optical absorption at the excitation wavelength. This heating will in turn produce significant pressures known as photoacoustic initial pressures through thermo-elastic expansion following p₀≈ηₜₕ Γϕμₐ where ηₜₕ is a conversion efficiency factor, Γ is a material property known as the Grüneissen parameter, ϕ is the focal fluence of the excitation beam and μₐ is the optical absorption of the medium at the given excitation wavelength. These pressures can be substantial, easily surpassing 100 MPa for excitation pulses within the ANSI optical exposure limits. These nontrivial pressures can produce modulations δn in the local refractive-index n₀ through the elasto-optic effect following n*=n₀+δn=n₀+(∈n₀³p₀)/(2ρvₛ²), where n* is the new refractive-index profile, ∈ is the elasto-optic coefficient, ρ is the mass density, and vₛ Is the acoustic propagation velocity. In certain PARS imaging embodiments, these refractive-index modulations can be measured using a continuous-wave interrogation beam which is co focused to the excitation location. This is detected as a total intensity measured on a photodiode such that all phase information from the interrogation spectrum is rejected. This can be simply represented as a change in reflectivity ΔR from the interrogation location which will be the difference between the perturbed reflectivity

$${R}^{*} = {\vert \frac{{n}_{1} + δ n - {n}_{2}}{{n}_{1} + Δ n + {n}_{2}} \vert}^{2} ,$$

and the unperturbed reflectivity

$$R = {\vert \frac{{n}_{1} - {n}_{2}}{{n}_{1} + {n}_{2}} \vert}^{2}$$

between the two media n₁, and n₂ such that for small perturbations δn (which are themselves proportional to the optical absorption μₐ) we get the approximate relationship ΔR ∝δn(n₁-n₂ (Haji Reza et al., Light: Science Applications volume 6, page 16278 (2017), the entirety of which is incorporated by reference herein). One interpretation of this result is that the intensity reflectivity from an excited interface relates directly to the inherent scattering contrast (n₁-n₂) and to the optical absorption.

In CEPARS, it may be desirable to exclude signals which have originated far from the focus. Previously, with conventional PARS embodiments, the axial characteristics were solely provided by the optical section defined by the focusing optics. However, it was found experimentally that axial performance can easily be far worse than this value. To improve this, CEPARS may add low-coherence interferometry such that signals which have originated from a path length significantly longer or shorter (defined by the coherence length of the interrogation source) than the reference path length will be excluded. In other words, signals which have originated from a path length that is more than a threshold amount different than the reference path length may be excluded. However, this will lead to ambiguity within the received signal as the two paths may still provide a signal which has undergone some amount of deconstructive interference. To combat this, CEPARS captures several (at least two) low-coherence interferometry signals which involve different reference path lengths. One example would be to compare half of the sample signal with one reference path, and the other half of the sample signal with a reference path where the phase has been offset by π/2. For complete characterization of the received signal, at least four components with appropriate phase offset such as of 0, π/2, π, and 3π/2 are required following from quadrature interferometry. This would allow for extraction of both an in-phase and quadrature signal simultaneously by rejecting undesired self-interference effects and reference-path signals such that phase-derived ambiguity can be eliminated.

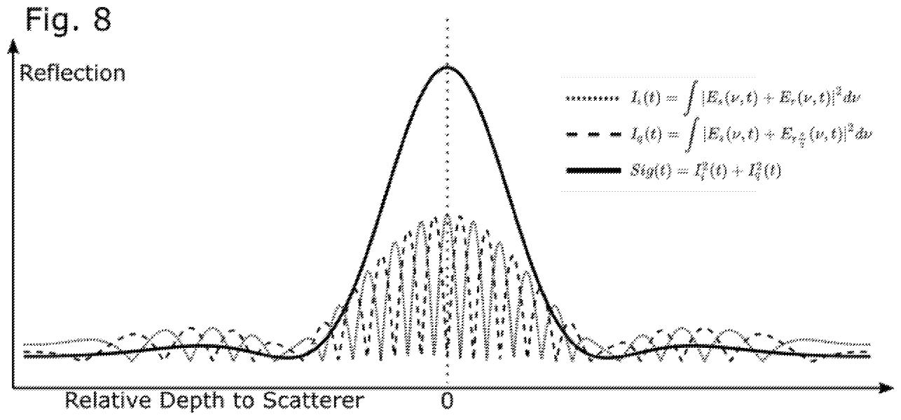

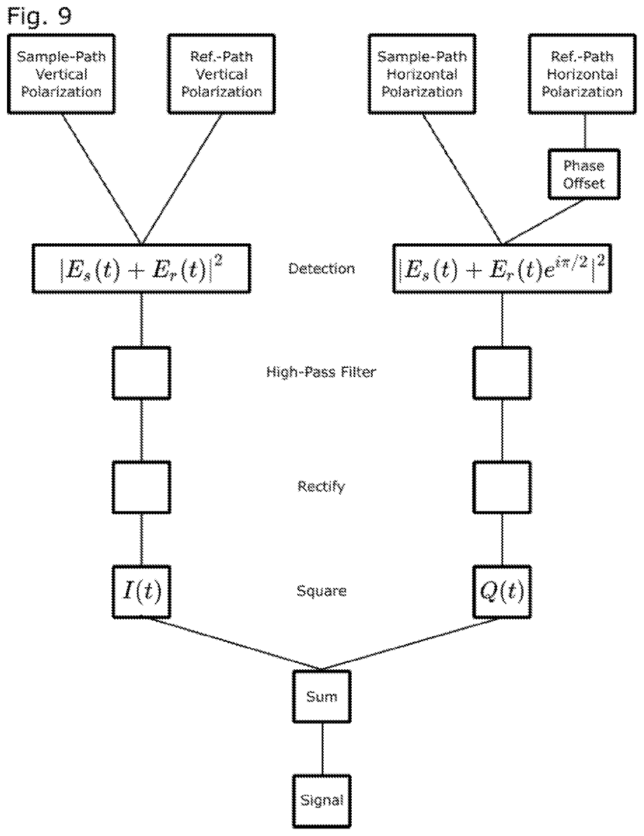

FIG. 8 (CEPARS Signals) shows an example of the above described signals. If we assume a single optical scatterer is placed at some location in the sample path (Eₛ (t, v)), and the mean reference path length is scanned about that same distance then the following two signals are acquired: for interference between reference path 1 (Eᵣ(t, v)) (no added delay) the corresponding measured intensity signal would be processed to provide Iᵢ (t)≈∫|Eₛ+Eᵣ|²dv-I_{c} likewise, for interference between reference path 2 (Eᵣ₂ (t, v)) (π/2 added delay) the corresponding measured intensity spiral would be processed to provide I_{q} (t)≈∫|Eₛ+Eᵣ₂|²dv-I_{c} where I_{c} is a calibration intensity, v is the optical frequency and Eₛ, Eᵣ, Eᵣ₂ are considered to have wide spectral content. Note that this is approximate as it assumes small self-interference effects within the sample. These signals then undergo high-pass filtering to remove the remaining mean signal offset, they are rectified, then finally their squares are summed producing the final time-domain signal Sig(t). These steps are highlighted in FIG. 9. To capture Sig(t) without approximation or calibration, a full-quadrature detection may be implemented providing a measure for example of I₀, I_{π/2}, I_{π}, I_{3π/2} which corresponds to sample-reference path delays of 0, π/2, π, and 3π/2 respectively. From here the complete low-coherence optical quadrature can be determined as Sig(t)=(I₀-I_{π})²+(I_{π/2}-I_{3π/2})². This process allows for acquisition of the low-coherence information within a rapid time scale. This is in contrast to other low-coherence methods such as time-domain optical-coherence tomography (TD-OCT) which commonly may necessitate axial scanning across the scatterer to properly characterize the sample. Such a TD-OCT approach would be ineffective for capturing the PARS mechanism largely due to issues involving acquisition time and repeatability concerns.

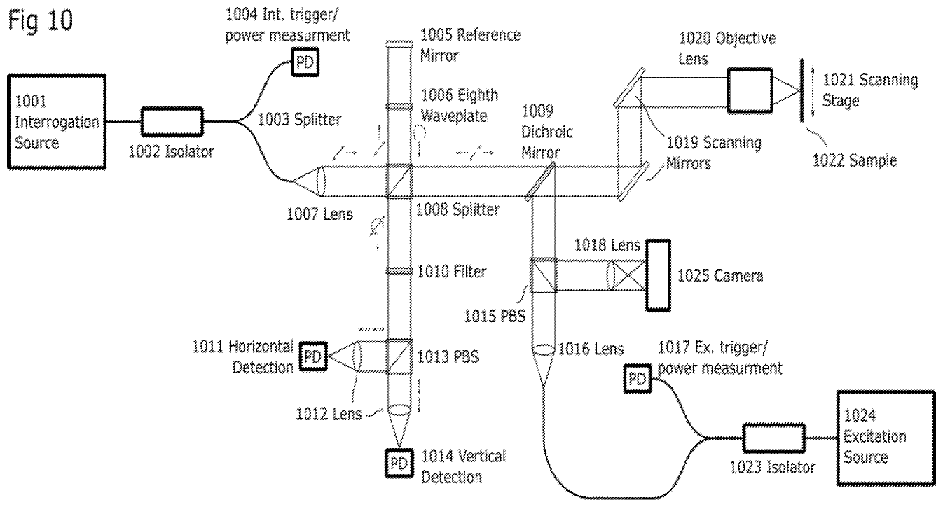

FIG. 10 highlights one possible implementation of CEPARS. A polarized interrogation source (1001) is fed into a beam-splitter (1008) which directs a portion of the beam towards the sample path and another portion towards the reference mirror (1005). The sample path of the interrogation is then combined with the excitation path using an appropriate dichroic mirror (1009). The two beams are then directed onto the sample (1022) using a set of scanning mirrors (1019) and an objective lens (1020). Here, scanning can also be performed using a mechanical scanning stage (1021) to overcome field of view limitations of the objective. The reference path passes through an eighth wave plate (1006) twice providing a circular polarized state where the total path length is controlled by position of the reference mirror. This circular polarized state will inherently provide the two desired reference phases. The linear polarized sample path returning from the sample is then combined with the circular reference path at the beam-splitter. Excess excitation light which has been transmitted through the dichroic mirror is further rejected by the use of a narrow filter (1010). Finally the two polarization states are split using a polarized beam-splitter (1013) and individual detection is then performed. Since this device will inherently be sensitive to polarization-dependent scattering in the sample, it may also be necessary to first characterize the given interrogation location with the reference path blocked such that the relative received values can be appropriately adjusted.

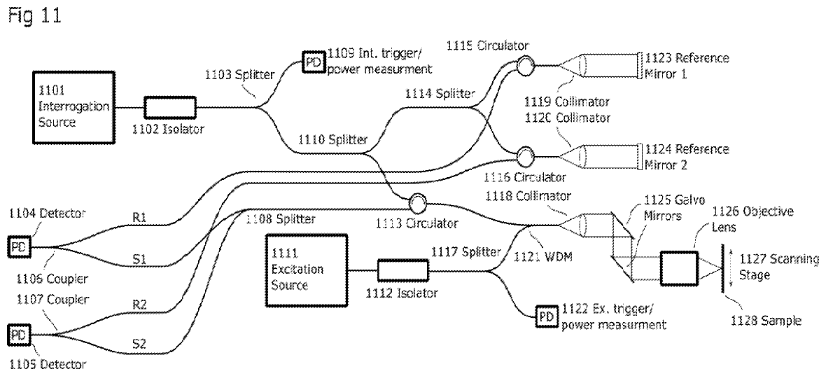

FIG. 11 highlights another possible implementation of CEPARS. This implementation features primarily fiber-based optics and takes advantage of a randomly polarized interrogation source to avoid polarization-dependent sensitivity at the sample. The interrogations source (1101) is split (1110) between the reference and sample pathways as before. Here, the reference path is further split (1114) to provide the desired added phase offsets. Polarization-independent circulators (1113, 1115, 1116) then redirect the reference paths (R1, R2) towards respective beam couplers (1106, 1107) where they are combined with the sample path components (S1, S2).

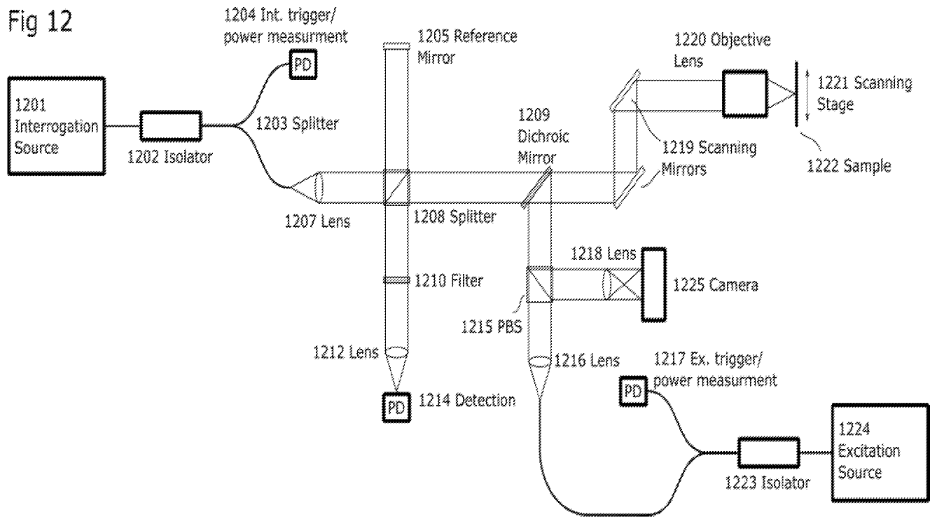

FIG. 12 highlights another possible implementation of CEPARS. This implementation features a serial acquisition as opposed to those represented and FIGS. 10 and 11, which utilize a parallel capture. Serial CEPARS only may necessitate a single low-coherence interferometer, but may require multiple acquisitions. Moreover, subsequent acquisitions must be performed with a varied reference path-length. For example, a dual acquisition might take one acquisition with a π/2 phase offset relative to a first acquisition provided by a piezo-mounted mirror (1205). In this manner, the in-phase and quadrature data can still be captured.

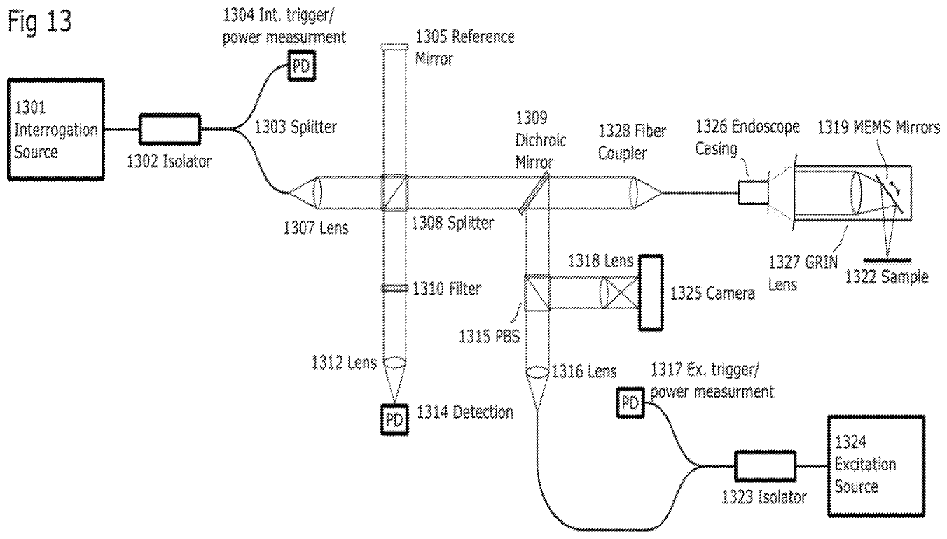

FIG. 13 highlights yet another possible implementation of CEPARS. This implementation features a serial acquisition as that presented in FIG. 12. However, rather than focusing directly onto a sample with free-space optics, here of the excitation and sample-path of the interrogation beams are coupled into a fiber which is fed through an endoscopic probe. At the distal end, optical focusing is provided by a GRIN lens (1327), and optical scanning is provided by a set of MEMS mirrors (1319). This represents a compact implementation capable of accessing challenging locales.

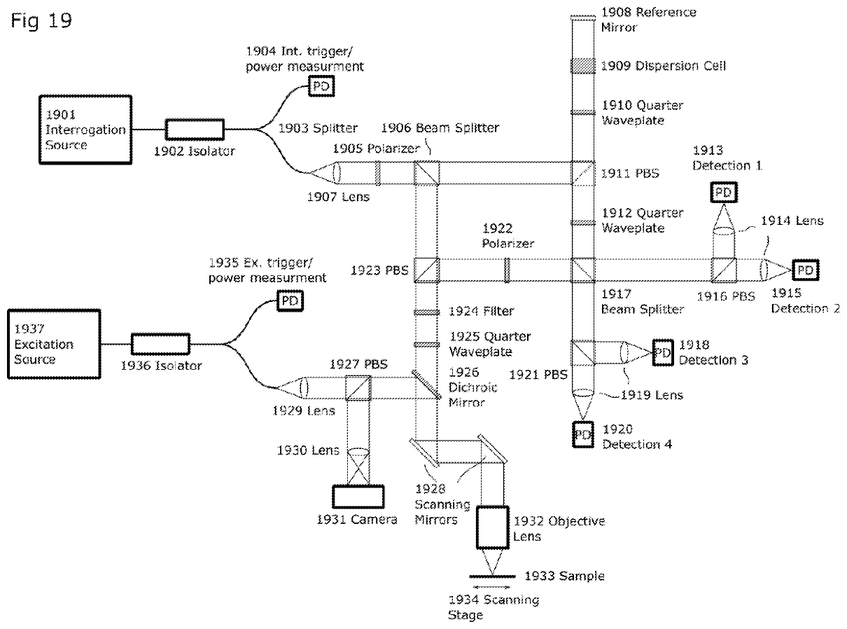

FIG. 19 highlights yet another possible implementation of CEPARS. This implementation utilizes a full optical-quadrature detection path. Unlike other and simpler described architectures, this embodiment may not require additional calibration, may not require assumption of small self-interference terms, and may not require multiple acquisition events providing more complete characterization of the tissue. The detection pathways include an interrogation source (1901), which is polarized (1905) and split (1903). The sample path transmits through a polarization-sensitive splitter (1923), is circularly polarized (by a quarter waveplate 1925), combined with the excitation pathway (at dichroic mirror 1926) and directed to the sample. The back-reflected portion is converted back to a linear polarization state (at quarter waveplate 1925), has remaining excitation removed by a filter (1924) and the light is again passed through a linear polarizer (1922) to ensure a clean polarization state. The reference path consists of a similar non-reciprocal pathways using a quarter-wave plate (1910) and PBS (1911). A dispersion cell (1909) can be added to compensate for sample-path dispersion. The length of this path can be controlled by changing the reference mirror (1908) position for appropriate depth selection within the sample. This light is circularly polarized (by quarter waveplate 1912) contributing a π/2 phase shift along one axis and recombined with the sample pathway in a non-polarizing splitter (1917). These two paths which are composed to multiple polarization states are further separated in two PBSs (1916, 1921) yielding the desired combinations of sample-path and reference-path phase delays for full-quadrature detection across four sensors (1913, 1915, 1918, 1920). The sensors 1913, 1915, 1918, and 1920 may be optical sensors, such as, e.g., a single photodiode, array of photodiodes, CCD etc. Then, the collected data will be processes to extract the PARS modulation quadrature information.

In some embodiments of SDCG-PARS, one goal is to provide a frill depth-resolved optical absorption profile of a sample without necessitating axial optical scanning. Conceptually, this is similar to how SD-OCT is operated. However the techniques are highly distinct from each other. First, it is assumed that the optical section can be considered a collection of ideal reflectors at some spatial distribution (along the z direction) such that is can be represented as rₛ(z). By interrogating the sample with a range of optical frequencies, commonly implemented as either a swept source or a stationary broadband spectrum source, a respective reflection spectrum can be collected. This involves combining the back-reflected light from the sample with a .reference such that the interference fringes now encode the locations of the optical scatters within the optical section. Recovery of the spatial reflection distribution then simply involves performing a frequency transform on the collected spectrum. Since the PARS mechanism involves modulation of the optical scattering properties within a sample where these modulations correspond to locations of optical absorption, by comparing the distribution of scatters both before and directly after photoacoustic excitation by an execution pulse, the difference at a given location will now correspond to the PARS-modulated regions which are optically absorbing. However, although high-bandwidth detectors are ideal for such a task they may prove highly impractical for implementation, and as such there is a requirement for a means of providing these two distinct interrogations. One proposed method is the use of a short (<100 ns) pulsed, or modulated interrogation laser which can effectively force a short interrogation time on a lower bandwidth detector such as a CCD array by reducing the amount of time back-reflected light from the sample will be incident on the array. This method allows for proper control over the relative timing of the excitation and interrogation pulses and the duration of the interrogation time.

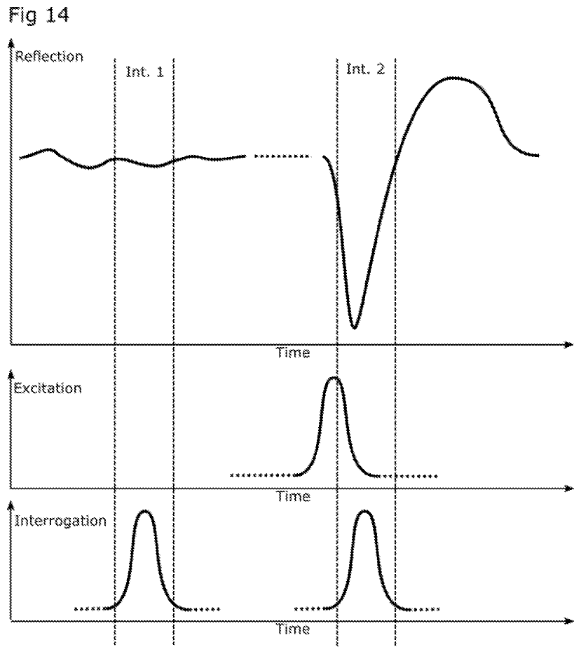

FIG. 14 shows an example of the relative timing between the reflectivity properties of a given wavelength in the sample, and the excitation and interrogation pulses. The second interrogation pulse which corresponds to be excited sample, must be timed such that it takes full advantage of the perturbed sample. This exact timing will vary significantly given all the available parameters such as the sample under consideration, the time evolution characteristics of the excitation, and the time revolution characteristics of the interrogation. In general, the rising edge of the interrogation will be less than 1 μs from the rising edge of the excitation. The duration of the interrogation pulse will also be less than 1 μs.

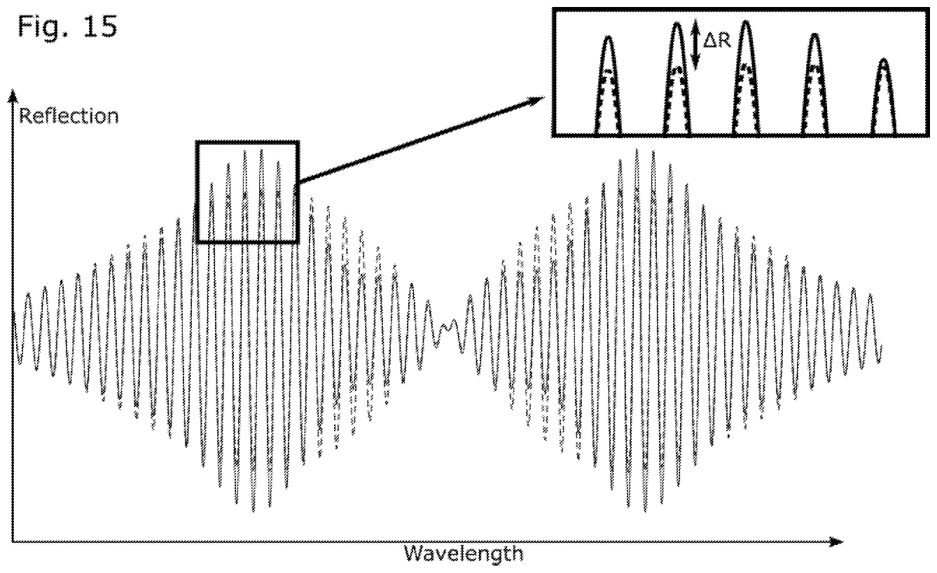

FIG. 15 shows an example of two collected spectra. One of the spectra is associated with an unperturbed interrogation event, the other is associated with an excited interrogation event. The small differences Δn between the spectra are associated with the PARS-modulated regions.

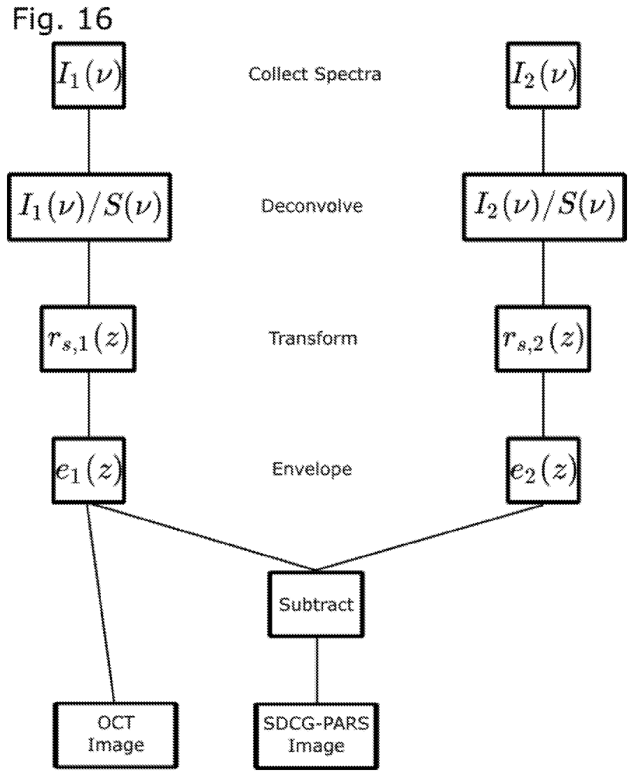

FIG. 16 shows a flow chart of the collection and processing involved with SDCG-PARS. The two collected spectra are first deconvolved with the original spectral content S(v). Here, other processing steps may be taken to reduce the effects of noise and other non-desirable effects. The spectra are then transformed back into a physical distribution representing the relative strength of optical scattering at a given depth rₛ(z). The envelope of each scattering distribution is taken, then the two envelopes are subtracted from each other to form a SDCG-PARS A-line. One of the two original envelopes can also be used to produce a conventional SD-OCT A-line.

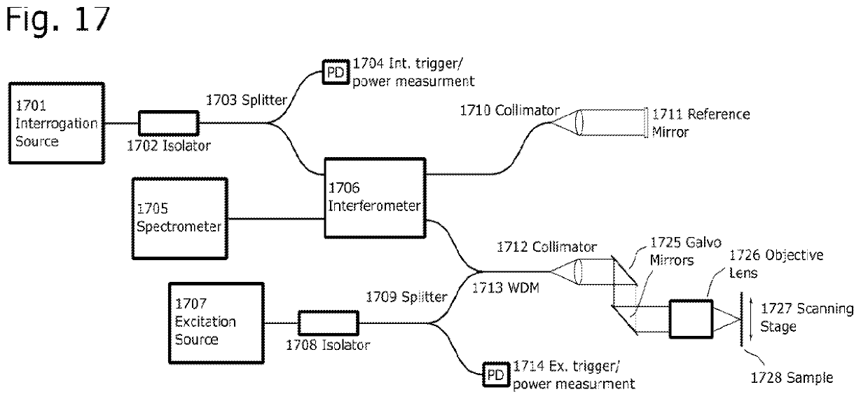

FIG. 17 shows an example system of a fiber-based SDCG-PARS. A pulsed interrogation source (1701) is split (by a splitter 1703) such that a portion is collected at a detector (1704) to characterize pulse-to-pulse consistency. The other portion is split into a sample path and a reference path. The reference path is directed on a reference mirror (1711) such that the total path length will be appropriately similar to the total sample path length facilitating low-coherence interferometry. The sample path is combined with the pulsed excitation source through a multiplexer (1713). The two beams are then scanned along the surface of the sample with a set of galvanometer mirrors (1725) and an appropriate objective lens (1726). The back-reflected light from the reference path and the sample path are then combined in a fiber coupler (1706) such that they interfere with each other. This resulting light is then fed into a CCD-based spectrometer (1705) for detecting of the spectra.

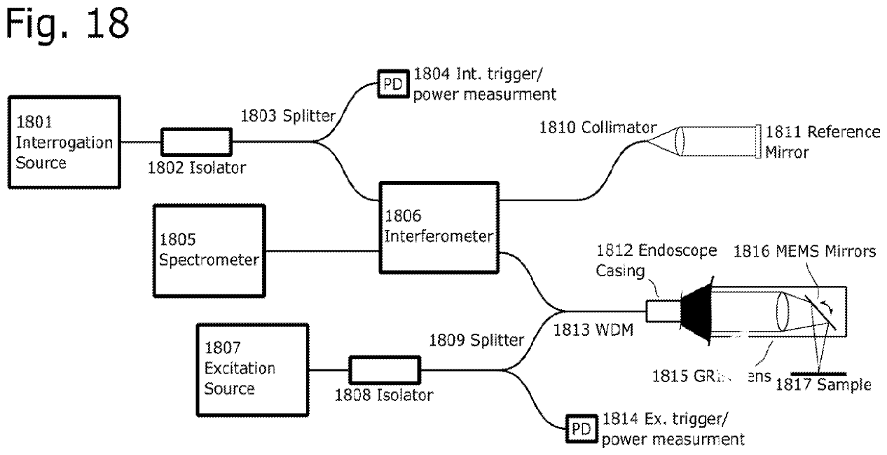

FIG. 18 shows another example of a SDCG-PARS, here with an endoscopic implementation. The primary difference between this and the previous embodiment is that after the multiplexer (1813), the combined beams are fed into an endoscopic casing (1812). Positioning of the final focus is now controlled by the use of a GRIN lens (1815) at the distal end of the fiber which is focusing through a MEMS mirror (1816) which provides lateral scanning of the interrogation spot on the sample (1817).

It will be apparent that other examples may be designed with different components to achieve similar results. Other alternatives may include various coherence length sources, use of balanced photodetectors, interrogation-beam modulation, incorporation of optical amplifiers in the return signal path, etc.

During in vivo imaging experiments, no agent or ultrasound coupling medium are required. However the target can be prepared with water or any liquid such as oil before non-contact imaging session. No special holder or immobilization is required to hold the target during imaging sessions.

Other advantages that are inherent to the structure will be apparent to those skilled in the art. The embodiments described herein are illustrative and not intended to limit the scope of the claims, which are to be interpreted in light of the specification as a whole.

The excitation beam may be any pulsed or modulated source of electromagnetic radiation including lasers or other optical sources. In one example, a nanosecond-pulsed laser was used. The excitation beam may be set to any wavelength suitable for taking advantage of optical (or other electromagnetic) absorption of the sample. The source may be monochromatic or polychromatic.

The interrogation beam may be any pulsed, or modulated source of electromagnetic radiation including lasers or other optical sources. Any wavelength can be used for interrogation purpose depending on the application.

CG-PARS may use any interferometry designs such as common path interferometer (using specially designed interferometer objective lenses), Michelson interferometer, Fizeau interferometer, Ramsey interferometer, Fabry-Perot interrometer, Mach-Zehnder interferometer, and optical-quadrature detection. The basic principle is that phase (and maybe amplitude) oscillations in the probing receiver beam can be detected using interferometry and detected at AC, RF or ultrasonic frequencies using various detectors.

In one example, both excitation and receiver beams may be combined and scanned. In this way, photoacoustic excitations may be sensed in the same area as they are generated and where they are the largest. Other arrangements may also be used, including keeping the receiver beam fixed while scanning the excitation beam or vice versa. Galvanometers, MEMS mirrors, polygon scanners, and stepper/DC motors may be used as a means of scanning the excitation beam, probe/receiver beam or both.

The excitation beam and sensing/receiver beam can be combined using dichroic minors, prisms, beam splitters, polarizing beam splitters etc. They can also be focused using different optical paths.

The reflected light may be collected by photodiodes, avalanche photodiodes, phototubes, photomultipliers, CMOS cameras, CCD cameras (including EM-CCD, intensified-CCDs, back-thinned and cooled CCDs), etc. The detected signal may be amplified by an RF amplifier, lock-in amplifier, trans-impedance amplifier, or other amplifier configuration. Also different methods may be used in order to filter the excitation beam from the receiver beam before detection. CG-PARS may use optical amplifiers to amplify detected light.

A table top, handheld, endoscopic, surgical microscope, or ophthalmic CG-PARS system may be constructed based on principles known in the art. CG-PARS may be used for A-, B- or C-scan images for in vivo, ex vivo or phantom studies.

CG-PARS may be optimized in order to take advantage of a multi-focus design for improving the depth-of-focus of 2D and 3D OR-CG-PARS imaging. The chromatic aberration in the collimating and objective lens pair may be harnessed to refocus light from a fiber into the object so that each wavelength is focused at a slightly different depth location. Using these wavelengths simultaneously may be used to improve the depth of field and signal to noise ratio (SNR) of CG-PARS images. During CG-PARS imaging, depth scanning by wavelength tuning may be performed.

The CG-PARS system may be combined with other imaging modalities such as fluorescence microscopy, two-photon and confocal fluorescence microscopy, Coherent-Anti-Raman-Stokes microscopy, Raman microscopy, Optical coherence tomography, other photoacoustic and ultrasound systems, etc. This systems could be combined by designing an optical combiner to integrate the optical paths of each systems. Also a processor to synchronise the results if necessary and analyse the results either separately or in combination. These integrated modalities can bring complementary imaging contrast. This could permit imaging of the microcirculation blood oxygenation parameter imaging, and imaging of other molecularly-specific targets simultaneously, a potentially important task that is difficult to implement with only fluorescence based microscopy methods. A multi-wavelength visible laser source may also be implemented to generate photoacoustic signals for functional or structural imaging.

Polarization analyzers may be used to decompose detected light into respective polarization states. The light detected in each polarization state may provide information about ultrasound-tissue interaction.

Applications

It will be understood that the system described herein may be used in various ways, such as those purposes described above, and also may be used in other ways to take advantage of the aspects described above. A non-exhaustive list of applications is discussed below.

The system may be used for imaging angiogenesis for different pre-clinical tumor models.

The system may also be used for clinical imaging of micro- and macro-circulation and pigmented cells, which may find use for applications such as in (1) the eye, potentially augmenting or replacing fluorescein angiography; (2) imaging dermatological lesions including melanoma, basal cell carcinoma, hemangioma, psoriasis, eczema, dermatitis, imaging Mohs surgery, imaging to verify tumor margin resections; (3) peripheral vascular disease; (4) diabetic and pressure ulcers; (5) burn imaging; (6) plastic surgery and microsurgery; (7) imaging of circulating tumor cells, especially melanoma cells; (8) imaging lymph node angiogenesis; (9) imaging response to photodynamic therapies including those with vascular ablative mechanisms; (10) imaging response to chemotherapeutics including anti-angiogenic drugs; (11) imaging response to radiotherapy.

The system may be useful in estimating oxygen saturation using multi-wavelength photoacoustic excitation and CG-PARS detection and applications including: (1) estimating venous oxygen saturation where pulse oximetry cannot be used including estimating cerebrovenous oxygen saturation and central venous oxygen saturation. This could potentially replace catheterization procedures which can be risky, especially in small children and infants.

Oxygen flux and oxygen consumption may also be estimated by using CG-PARS imaging to estimate oxygen saturation, and an auxiliary method to estimate blood flow in vessels flowing into and out of a region of tissue.

The system may also have some gastroenterological applications, such as imaging vascular beds and depth of invasion in Barrett's esophagus and colorectal cancers. Depth of invasion is key to prognosis and metabolic potential. Gastroenterological applications may be combined or piggy-backed off of a clinical endoscope and the miniaturized CG-PARS system may be designed either as a standalone endoscope or fit within the accessory channel of a clinical endoscope.

The system may have some surgical applications, such as functional imaging during brain surgery, use for assessment of internal bleeding and cauterization verification, imaging perfusion sufficiency of organs and organ transplants, imaging angiogenesis around islet transplants, imaging of skin-grafts, imaging of tissue scaffolds and biomaterials to evaluate vascularization and immune rejection, imaging to aid microsurgery, guidance to avoid cutting critical blood vessels and nerves.

Other examples of applications may include CG-PARS imaging of contrast agents in clinical or pre-clinical applications; identification of sentinel lymph nodes; non- or minimally-invasive identification of tumors in lymph nodes; imaging of genetically-encoded reporters such as tyrosinase, chromoproteins, fluorescent proteins for pre-clinical or clinical molecular imaging applications; imaging actively or passively targeted optically absorbing nanoparticles for molecular imaging; and imaging of blood clots and potentially staging the age of the clots.

In some embodiments, any suitable technology, such as, e.g., OCT, can be used for surface topology (for constant- or variable-depth focusing kw photoacoustic remote sensing technologies) before imaging with CG-PARS.

In at least some embodiments, systems of the present disclosure may include variable-focal-length lenses (including voice-coil-driven, MEMS-based, piezoelectric-based, and tunable acoustic gradient lenses). Furthermore, systems of the present disclosure may include double-clad fiber couplers for both OCT and PARS microscopy (including CG-PARS) to deliver excitation light (and/or interrogation light) from a single-mode fiber to the sample, but collect interrogation light using the multi-mode cladding of the double-clad fiber. Systems of the present disclosure also may be used with angiography or Doppler.

Embodiments of the present disclosure may include one or more of the following advantages:
1. The proposed CG-PARS provides depth-dependent contrast which is directly proportional to optical absorption of the excitation laser. For example, CW CE-PARS extracts modulated components of signals using high-pass or bandpass filters. Pulsed detection systems associated with Pulsed CE-PARS or SD-CG-PARS uses differences in detected signals with and without excitation pulses.
2. The coherence length of the source is preferably shorter than the depth-of-focus of the interrogation beam into the sample, and more preferably significantly shorter. In this way, improved depth resolution can be achieved by use of coherence-gating.
3. The proposed SD-CG-PARS system incorporates a spectrometer and is able to detect enveloped A-scans with and without excitation pulses (or with different pulse energies). The system uses a processor for extracting differences in the enveloped A-scans with and without excitation pulse (or with different pulse energies).
4. In the proposed CE-PARS system, there may be two or more interferometers, or a method for sequentially interrogating with two or more successive reference path phase shifts and a processor for combining serial or parallel acquisitions to extract temporal modulations of the envelope signal.
5. The proposed CG-PARS methods uses OCT signals to detect refractive index changes associated with initial pressures and uses at least two acquisitions (either in serial or parallel with multiple detectors). In SD-CG-PARS, an A-scan OCT envelope acquisition is obtained with and without and excitation pulse, where each A-scan is acquired with a spectrometer. In CE-PARS we in-phase and quadrature components of the signal are acquired.
6. As noted, the SD-CG PARS method uses a spectrometer. Additionally, SD-CG-PARS may be used to detect enveloped OCT A-scans with and without excitation pulses (or with different pulse energies). The phase in the detected signal may be removed to form an envelope. For SD-CG-PARS, a processor may be used to extract differences in the enveloped A-scans with and without excitation pulses (or with different pulse energies).

Certain examples of remote sensing systems may be described as follows:

1. A Spectral-Domain Coherence-Gated PARS Tomogaphy (SD-CG-PARS Tomography) system having:
a. A pulsed excitation electromagnetic radiation source
b. A low-coherence interrogation light source, the coherence length being the principal determinant of the depth resolution. Typically, the interrogation wavelengths and the excitation wavelengths are spectrally distinct, but in an optional embodiment, the excitation and interrogation sources could be one and the same.
c. A combiner to combine the pulsed excitation beam and the interrogation beam to enable co-scanning of both beams
d. Focusing lens(es) for focusing the respective or combined beams and for collecting interrogation light from the sample.
e. An interferometer, having a splitter to split the interrogation beam into a reference path and a signal path, the reference path having an adjustable path-length, and the signal path returning collected signals back to interfere with reference path light.
f. A light analysis module consisting of a spectrometer (with various types of dispersive elements: gratings, prisms, etc) and detector arrays (CCD, CMOS, photodiode array).
g. A temporal gating system to ensure that optical signals recorded after the excitation pulses are read out within a short (<tens of nanoseconds or <1us) time-scale before acoustic waves propagate far from their origin. Specifically, the acoustic distance-of-propagation over the interrogation readout time should not be significantly greater than the desired axial or lateral spatial resolution. This temporal gating could be accomplished using (1) a (fs-us-scale pulsed interrogation source and pulse-sequencer and acquisition electronics carefully timed to read out signals within nanoseconds after the excitation source. (2) an optical or electronic shutter with nanosecond-scale response times to enable the capture of ONLY the interrogation light within the desired temporal window (3) fast photodiode array to electronically capture time domain signals from each element and capturing only the first T time samples.
h. A pulse sequencer and acquisition system for forming at least two OCT A-scan lines per scan position: one with an excitation pulse and one without an excitation pulse OR one with a different excitation pulse energy than another.
i. Optional reference photodiode measurement subsystem to account for pulse-to-pulse variations of the excitation source and for variations in the interrogation source.
j. Optional programmable controller and actuator to adjust the reference pathlength between scans or to adjust the desired depth-sectioning.
k. Optional filter to reject excitation laser wavelengths from being detected by the spectrometer detectors.
l. A processor for processing the OCT RF A-scan lines (with- and without excitation pulses or with differing pulse energies and optionally with and without reference pathlength shifts) to form CG-PARS A-Scans with contrast proportional to optical absorption at each depth position. One such processor embodiment includes forming the envelope of each OCT A-scan and subtracting the envelopes of A-scans with and without excitation laser pulses. This strategy has the advantage of eliminating unwanted phase-noise sensitivity but will still capture refractive index changes associated with photoacoustic initial pressures.

m. A processing system to render and display OCT and CG-PARS images

2. A coherence-enhanced PARS (CE-PARS) microscopy system having:
a. A pulsed excitation light source
b. A low-coherence interrogation laser, the coherence length being the principal determinant of the depth resolution. Typically, the interrogation wavelengths and the excitation wavelengths are spectrally distinct, but in an optional embodiment, the excitation and interrogation sources could be one and the same.
c. A combiner to combine the pulsed excitation beam and the interrogation beam to enable co-scanning of both beams
d. Focusing lens(es) for focusing the respective or combined beams and for collecting interrogation light from the sample.
e. An interferometer, having a splitter to split the interrogation beam into a reference path and a signal path, the reference path having an adjustable path-length, and the signal path returning collected signals back to interfere with reference path light.
f. Light detection module(s) including associated optional amplifiers and filters, for example, consisting of photodiode(s) or balanced photodiode(s). Filters may be included to reject DC scattered light and collect only the modulated component in the case of CW interrogation beams. See description of module for pulsed interrogation light below.
g. A method for acquiring effective inphase- and quadrature complex envelope signals from the interfering light using one of two methods (1) serially, by performing a point-scan, lateral-scan, depth-scan or C-scan then adjusting the reference pathlength by π/2 phase then scanning again. (2) in parallel by using an additional interferometer with a reference path differing by π/2 from the reference path of the other interferometer. This parallel interferometer may be implemented with separate optical paths or as a common-path configuration. Note that this quadrature-sampling scheme offers the flexibility of C-scanning or en-face scanning at a particular depth gating (or depth range) without requiring acquisition of complete depth-scans (A-scans) to create an effective PARS image within a precise depth-section. If an A-scan is acquired, there mast be an excitation pulse for every depth sample in the A-scan line, which could lead to unwanted persistent laser exposure compared to the scanned beam case.
h. A processor for estimating the envelope or specifically, the magnitude of the complex envelope signal for cases with and without an excitation pulse (or with excitation pulses of different strengths).
i. A processor for processing the OCT RF envelope signals (with- and without excitation pulses or with differing pulse energies) to form CE-PARS signals with contrast proportional to optical absorption at each scan position. One such processor embodiment includes forming the envelope of each OCT signal and subtracting the envelopes with and without excitation laser pulses. This strategy has the advantage of eliminating unwanted phase-noise sensitivity but will still capture refractive index changes associated with photoacoustic initial pressures.
j. A temporal gating system to ensure that optical signals recorded after the excitation pulses are read out within a short (<tens of nanoseconds) time-scale before acoustic waves propagate far from their origin. Specifically, the acoustic distance-of-propagation over the interrogation readout time should not be significantly greater than the desired axial or lateral spatial resolution. This temporal gating could be accomplished using (1) a nanosecond-scale pulsed interrogation source and pulse-sequencer and acquisition electronics carefully timed to read out signals within nanoseconds after the excitation source. (2) an optical or electronic shutter with nanosecond-scale response times to enable the capture of ONLY the interrogation light within the desired temporal window (3) acquiring the photodiode signal as a function of time then sampling only the first few tens to hundreds of ns OR (4) using an analog or digital peak detector to extract the peak (envelope) signal or peak-to-peak (envelope) signal for each pulse.
k. A processing system to render and display OCT and CG-PARS images.

3. A pulsed interrogation detection subsystem which involves capturing an interrogation pulsed signal from the sample (with or without reference beam interference) both with or without an excitation pulse (or with differing pulse energies) and subtraction of the respective signals or estimating their relative difference normalized to the OCT signal without an excitation pulse present. This can be done by recording amplified photodiode signals with an analog-to-digital converter and doing the subtraction (and optionally division) operations digitally. It can also be done with analog electronics
4. A functional imaging system involving sequential pulses using (1) different excitation wavelengths or (2) different pulse widths (e.g. ps pulses and us pulses). In the case of both (I) and (2) the PARS initial pressure signals are proportional to optical absorption and detected optically using interrogation beams using the CG-PARS systems described above, or using previously described interferometric or non-interferometric PARS systems.

Examples of methods of for remote sensing may be described as follows:
a. (SDCG-PARS) A method for interrogating the optical properties of a sample which comprises:
   A method of generating photoacoustic signals within a sample;
   A low-coherence interferometer used to detect photoacoustic signals;
   A method for directing light towards a sample at a given location;
   A method of collecting light from a sample at a given location;
   An optical spectrum detector;
   A processor for collecting multiple optical spectra; and
   A processor for extracting differences between multiple optical spectra.
      I. The method of statement a., wherein the method of generating photoacoustic signals within a sample comprises a narrowband or broadband electromagnetic source which is one of a pulsed source, or a continuous-wave source which is intensity modulated.
         i. The method of I., wherein the portion of the excitation source is detected by photodiode to account for pulse-to-pulse variations.
      II. The method of statement a., wherein the low-coherence interferometer comprises a broadband electron source Which is one of a pulsed source, or a continuous-wave source which is intensity modulated, a method of splitting this beam into a reference path and a sample path, and a method of combining the beams returning from the reference path and the sample path.
         i. The method of statement II, wherein the optical spectrum detector comprises one or more dispersive elements (gratings, prisms, etc) and one or more detector arrays (CCD, CMOS, photodiode, etc.).
         ii. The method of statement II., wherein the portion of the interrogation source is detected by photodiode to account for pulse-to-pulse variations.
      III. The method of statement a, wherein the low-coherence interferometer comprises a broadband continuous-wave source electromagnetic source, a method of splitting this beam into a reference path and a sample path, and a method of combining the beams returning from the reference path and the sample path.
         i. The method of statement III, wherein the optical spectrum detector comprises one or more dispersive elements (gratings, prisms, etc) and one or more high-bandwidth detector arrays (photodiode, avalanche photodiode, etc.).
         ii. The method of statement III, wherein the portion of the interrogation source is detected by photodiode to account for power and variations.
      IV. The method of statement a., wherein the method for directing towards and from a sample comprises of an optical scanner (one or more of Galvanometer mirrors, resonant mirrors, MEMS mirrors, polygon seamier, etc.), focusing optic subsystem (objective lens, reflective objective, parabolic mirror, GRIN lens, and a system of optical filters to reject excitation wavelengths along the detection path.
      V. The method of statement a., wherein the method for directing towards and from a sample comprises of an light guide (optical fiber, double clad fiber, optical fiber bundle, etc.), an optical scanner (one or more of Galvanometer mirrors, resonant mirrors, MEMS mirrors, polygon scanner, etc.), and focusing optic subsystem (objective lens, reflective objective, parabolic minor, GRIN lens), and a system of optical filters to reject excitation wavelengths along the detection path.
      VI. The method of statement a, wherein the processors for collecting multiple optical spectra and for extracting differences between multiple optical spectra are implemented as electronic devices.
b. (Parallel CEPARS) A method for interrogating the optical properties of a sample which comprises:
   A method of generating photoacoustic signals within a sample;
   Two or more optical low-coherence interferometers used to detect photoacoustic signals;
   A method for directing light towards a sample at a given location;
   A method of collecting fight from a sample at a given location;
   A processor to combine data channels from the interferometers; and
   A processor to extract temporal modulations of the envelope signal.
      I. The method of statement b, wherein the method of generating photoacoustic signals within a sample comprises a narrowband or broadband electromagnetic source which is one of a pulsed source, or a continuous-wave source which is intensity modulated.
         i. The method of statement I, wherein the portion of the excitation source is detected by photodiode to account for pulse-to-pulse variations.
      II. The method of statement b, wherein the method for directing towards and from a sample comprises of an optical scanner (one or more of Galvanometer mirrors, resonant mirrors, MEMS mirrors, polygon scanner, etc.), focusing optic subsystem (objective lens, reflective objective, parabolic mirror, GRIN lens, and a system of optical filters to reject excitation wavelengths along the detection path.
      I. The method of statement b, wherein the method for directing towards and from a sample comprises of an light guide (optical fiber, double clad fiber, optical fiber bundle, etc.), an optical scanner (one or more of Galvanometer mirrors, resonant mirrors, MEMS mirrors, polygon seamier, etc.), and focusing optic subsystem (objective lens, reflective objective, parabolic mirror, GRIN lens), and a system of optical filters to reject excitation wavelengths along the detection path.
c. (serial QSCG-PARS) A method for interrogating the optical properties of a sample which comprises:
   A method of generating photoacoustic signals within a sample:
   A low-coherence interferometer used to detect photoacoustic signals where reference phase must be adjusted between sequential acquisitions;
   A method for directing light towards a sample at a given location;
   A method of collecting light from a sample at a given location;
   A method of acquisition which necessitates multiple acquisitions;
   A processor to combine serial data channels from the interferometer; and
   A processor to extract temporal modulations of the envelope
      I. The method of statement c, wherein the method of generating photoacoustic signals within a sample comprises a narrowband or broadband electromagnetic source which is one of a pulsed source, or a continuous-wave source which is intensity modulated.
         ii. The method of statement I, wherein the portion of the excitation source is detected by photodiode to account for pulse-to-pulse variations.
      II. The method of statement c, wherein the method for directing towards and from a sample comprises of an optical scanner (one or more of Galvanometer mirrors, resonant minors, MEMS mirrors, polygon scanner, etc.), focusing optic subsystem (objective lens, reflective objective, parabolic mirror, GRIN lens, and a system of optical filters to reject excitation wavelengths along the detection path.
      III. The method of statement c, wherein the method for directing towards and from a sample comprises of an light guide (optical fiber, double clad fiber, optical fiber bundle, etc.), an optical scanner (one or more of Galvanometer mirrors, resonant mirrors, MEMS mirrors, polygon scanner, etc.), and focusing optic subsystem (objective lens, reflective objective, parabolic mirror, GRIN lens), and a system of optical filters to reject excitation wavelengths along the detection path.
d. (Quadrature CEPARS) A method for interrogating the optical properties of a sample which comprises:
   A method of generating photoacoustic signals within a sample;
   An optical quadrature detector;
   A method for directing light towards a sample at a given location;
   A method of collecting light from a sample at a given location;
   A processor to combine data channels from the quadrature detector; and
   A processor to extract temporal modulations of the envelope signal.
      I. The method of statement d., wherein the method of generating photoacoustic signals within a sample comprises a narrowband or broadband electromagnetic source which is one of a pulsed source, or a continuous-wave source which is intensity modulated.
         i. The method of statement I, wherein the portion of the excitation source is detected by photodiode to account for pulse-to-pulse variations.
      II. The method of statement d., wherein the method for directing towards and from a sample comprises of an optical scanner (one or more of Galvanometer mirrors, resonant mirrors, MEMS mirrors, polygon scanner, etc.), focusing optic subsystem (objective lens, reflective objective, parabolic mirror, GRIN lens, and a system of optical filters to reject excitation wavelengths along the detection path.
      III. The method of statement d, wherein the method for directing towards and from a sample comprises of an light guide (optical fiber, double clad fiber, optical fiber bundle, etc.), an optical scanner (one or more of Galvanometer mirrors, resonant mirrors, MEMS mirrors, polygon scanner, etc.), and focusing optic subsystem (objective lens, reflective objective, parabolic minor, GRIN lens), and a system of optical filters to reject excitation wavelengths along the detection path.

1.-35. (canceled)

36. A coherence gated photoacoustic remote sensing system for imaging a portion of a sample with optical resolution, comprising:
one or more laser sources configured to generate at least one excitation beam that induces ultrasonic signals in the sample at an excitation location,
wherein the one or more laser sources are also configured to generate at least one interrogation beam incident on the sample at an interrogation location, a portion of the at least one interrogation beam returning from the sample that is indicative of the generated ultrasonic signals, the at least one interrogation beam;
an optical system configured to focus the at least one excitation beam onto the sample at an excitation location, and/or the at least one interrogation beam onto the sample, along a sample path, at an interrogation location, at least the interrogation location being below the surface of and within the sample; and
an interferometer configured to isolate a returning portion of the at least one interrogation beam that corresponds to an interrogation event of the sample.

37. The system of claim 36, further comprising a reference beam source configured to generate a reference beam that travels along a reference path, wherein the interferometer isolates the returning portion using the reference beam,
wherein the reference beam source is configured to generate one or more additional reference beams that are phase shifted relative to the reference beam, and wherein the interferometer isolates the returning portion using the reference beam and the one or more additional reference beams.

38. The system of claim 37, wherein the one or more additional reference beams are phased shifted by at least one of a different path length, one or more wave plates, or one or more circulators.

39. The system of claim 37, wherein the one or more additional reference beams are detected either in parallel or serially with the reference beam.

40. The system of claim 36, wherein at least one of the excitation beam or the interrogation beam is pulsed or intensity-modulated.

41. The system of claim 36, wherein at least one of the excitation location or the interrogation location is within 1 mm of the surface of the sample.

42. The system of claim 36, wherein at least one of the excitation location or the interrogation location is greater than 1 μm below the surface of the sample.

43. The system of claim 36, wherein the excitation location and the interrogation location are focal points that are at least partially overlapping.

44. The system of claim 36, further comprising at least one detector configured to collect the returning portion of the at least one interrogation beam.

45. The system of claim 36, further comprising a processor that calculates an image of the sample based on the returning portion of the interrogation beam.

46. The system of claim 36, wherein, for each detection location, the system applies an excitation beam with more than one frequency, bandwidth, phase shift, or combination thereof.

47. The system of claim 36, wherein the optical system interrogates each interrogation location in a non-excited state and after the excitation beam excites the sample.

48. The system of claim 36, wherein the one or more laser sources are configured to generate one or more excitation beams that excites the sample with a plurality of frequencies, a plurality of bandwidths or combinations thereof.

49. The system of claim 36, wherein the one or more laser sources include an excitation beam source configured to generate the excitation beam, and an interrogation beam source configured to generate the interrogation beam.

50. The system of claim 36, further including a fiber optic cable having an input end and a detection end, wherein the one or more laser sources are coupled to the input end.

51. The system of claim 36, further including a detector capable of detecting a spectral content of combined reference and sample paths, wherein the detector is configured to provide interrogation within 100 ns.

52. The system of claim 37, further including at least one optical combiner configured to compare the portion of the at least one interrogation beam returning from the sample with the reference beam.

53. The system of claim 52, further including a processing unit for interpreting the comparison between the portion of the at least one interrogation beam returning from the sample and the reference beam.

54. The system of claim 36, wherein the at least one laser source includes a pulsed interrogation source or a rapidly modulation continuous-wave source or is a swept source laser.

55. The system of claim 36, used for one or more of:
imaging angiogenesis for pre-clinical tumor models;
estimating oxygen saturation using multi-wavelength photoacoustic excitation;
estimating venous oxygen saturation where pulse oximetry cannot be used;
estimating cerebrovenous oxygen saturation and/or central venous oxygen saturation;
estimating oxygen flux and/or oxygen consumption;
estimating blood flow in vessels flowing into and out of a region of tissue;
clinical imaging of micro- and macro-circulation and pigmented cells;
imaging of the eye;
augmenting or replacing fluorescein angiography;
imaging dermatological lesions;
imaging melanoma;
imaging basal cell carcinoma;
imaging hemangioma;
imaging psoriasis;
imaging eczema;
imaging dermatitis;
imaging Mohs surgery;
imaging to verify tumor margin resections;
imaging peripheral vascular disease;
imaging diabetic and/or pressure ulcers;
burn imaging;
plastic surgery;
microsurgery;
imaging of circulating tumor cells;
imaging melanoma cells;
imaging lymph node angiogenesis;
imaging response to photodynamic therapies;
imaging response to photodynamic therapies having vascular ablative mechanisms;
imaging response to chemotherapeutics;
imaging response to anti-angiogenic drugs;
imaging response to radiotherapy;
imaging vascular beds and depth of invasion in Barrett's esophagus and/or colorectal cancers;
functional imaging during brain surgery;
assessment of internal bleeding and/or cauterization verification;
imaging perfusion sufficiency of organs and/or organ transplants;
imaging angiogenesis around islet transplants;
imaging of skin-grafts;
imaging of tissue scaffolds and/or biomaterials to evaluate vascularization and/or immune rejection;
imaging to aid microsurgery;
guidance to avoid cutting blood vessels and/or nerves;
imaging of contrast agents in clinical or pre-clinical applications;
identification of sentinel lymph nodes;
non- or minimally-invasive identification of tumors in lymph nodes;
imaging of genetically-encoded reporters, wherein the genetically-encoded reporters include tyrosinase, chromoproteins, and/or fluorescent proteins for pre-clinical or clinical molecular imaging applications;
imaging actively or passively targeted optically absorbing nanoparticles for molecular imaging;
imaging of blood clots;
staging an age of blood clots;
replacing a catheterization procedure;
gastroenterological applications;
single-excitation pulse imaging over an entire field of view;
imaging of tissue;
imaging of cells;
imaging of absorption-induced changes of scattered light; or non-contact imaging of optical absorption.

56. A system including the coherence gated photoacoustic remote sensing system of claim 36 in combination with fluorescence microscopy, two-photon and confocal fluorescence microscopy, Coherent-Anti-Raman-Stokes microscopy, Raman microscopy, or Optical coherence tomography.

57. The system of claim 36, wherein the system is configured to provide depth-dependent contrast, wherein the depth-dependent contrast is directly proportional to optical absorption of the excitation beam.

58. The system of claim 36, wherein a coherence length of the excitation beam is shorter than a depth-of-focus of the interrogation beam.

59. The system of claim 36, wherein the system is configured to use OCT signals to detect refractive index changes associated with initial pressures, wherein the system uses at least two acquisitions, either in serial or parallel with multiple detectors.

60. The system of claim 36, further including a spectrometer configured to acquire an A-scan with or without excitation pulses.

61. The system of claim 36, wherein the at least one interrogation beam is a low-coherence beam, or the interferometer is a low-coherence interferometer.

62. The system of claim 36, wherein the interrogation beam has pulses that are sufficiently short such that detection error introduced by acoustic propagation is negligible.
